(19) United States
(12) Patent Application Publication
(10) Pub. No.: US 2021/0189474 A1
(43) Pub. Date: Jun. 24, 2021

(54) DNA TESTING CHIP, DNA TESTING METHOD, AND DNA TESTING SYSTEM

(71) Applicant: NEC Corporation, Minato-ku, Tokyo (JP)

(72) Inventor: Kenji MIYAZAKI, Tokyo (JP)

(73) Assignee: NEC Corporation, Minato-ku, Tokyo (JP)

(21) Appl. No.: 16/076,356

(22) PCT Filed: Feb. 7, 2017
(86) PCT No.: PCT/JP2017/004323

§ 371 Date: Aug. 8, 2018

(30) Foreign Application Priority Data
Feb. 10, 2016 (JP) 2016-023542

(51) Int. Cl. C12Q 1/6837; C12Q 1/686

(52) U.S. Cl. CPC C12Q 1/6837; C12Q 1/686

(57) ABSTRACT

[Object] To provide a DNA testing chip, a DNA testing method, a DNA testing system, and a DNA testing device, being capable of performing a DNA test with a simplified processing process and a simplified processing mechanism. [Solution] A DNA testing chip comprises a chamber into which a PCR reaction solution is injected, and a sensor. The chamber comprises a region having a plurality of aligned spots on which a single-stranded DNA forms a solid phase. The plurality of aligned spots provided such that each have a different combination of the corresponding genetic locus and the number of repeats. The single-stranded DNA includes an STR sequence having the number of repeats and the genetic locus corresponding to each spot. The sensor is used for determining whether or not the single-stranded DNA on each spot forms a hydrogen bond with a complementary single-stranded DNA in the PCR reaction solution

### TECHNICAL FIELD

The present invention relates to a DNA testing chip, a DNA testing method, a DNA testing system, and a DNA testing chip control device.

### BACKGROUND ART

A deoxyribonucleic acid (DNA) testing technique used for identifying a suspect in criminal investigation and for a paternity test is known. For example, when identifying a suspect, it is determined whether or not numbers of repeats in short tandem repeat (STR) sequences in a plurality of genetic loci coincide with each other between DNA in a bloodstain remaining at a crime scene and DNA of the suspect. Further, a microchip for use in a DNA test has also been developed (PTL 1).

### CITATION LIST

### Patent Literature

[PTL 1] International Publication No. WO2009/119698

### SUMMARY OF INVENTION

### Technical Problem

The following analysis is made from a viewpoint of the present invention. Note that it is assumed that disclosure of the above-described prior art document is incorporated in the present specification by reference.

In the above-described DNA testing technique, it is necessary to perform polymerase chain reaction (PCR) and electrophoresis individually for each genetic locus in order to measure a number of repeats in an STR sequence of the respective genetic loci. This is because, when PCR and electrophoresis for a plurality of genetic loci are performed all at once (e.g. PCR is performed in one PCR tube, and electrophoresis is performed by using one capillary), it is not possible to specify from which genetic locus, a detection peak is derived. Unless being specified for each genetic locus, a number of repeats in an STR sequence is meaningless. In this way, in the above-described DNA testing technique, a complex processing process and a complex processing mechanism are required. There is a need for a technique of performing a DNA test with a simplified processing process and a simplified processing mechanism.

In view of the above, an object of the present invention is to provide a DNA testing chip, a DNA testing method, a DNA testing system, and a DNA testing device, being capable of performing a DNA test with a simplified processing process and a simplified processing mechanism.

### Solution to Problem

According to a first aspect of the present invention, a DNA testing chip described below is provided. The DNA testing chip comprises a chamber into which a PCR reaction solution is injected; and a sensor. The chamber comprises a region where a plurality of spots are aligned. A single-stranded DNA forms a solid phase on each of the spots. The plurality of spots are formed in such a way that combinations of a genetic locus and a number of repeats corresponding to the spot are different from one another. The single-stranded DNA comprises an STR sequence having a genetic locus and a number of repeats associated with each of the spots. The sensor is used for determining whether or not a complementary single-stranded DNA in the PCR reaction solution forms a hydrogen bond to a single-stranded DNA on each spot.

According to a second aspect of the present invention, a DNA testing method employing a DNA testing chip described below is provided. The DNA testing method comprises a step of preparing the DNA testing chip. The DNA testing chip comprises a chamber into which a PCR reaction solution is injected, and a sensor. The chamber comprises a region where a plurality of spots on each of which a single-stranded DNA forms a solid phase are aligned. The plurality of spots are formed in such a way that combinations of an associated genetic locus and an associated number of repeats are different from one another. The single-stranded DNA comprises an STR sequence having a genetic locus and a number of repeats associated with each of the spots. The sensor is used for determining whether or not a complementary single-stranded DNA in the PCR reaction solution forms a hydrogen bond to a single-stranded DNA on each spot. The DNA testing method further includes: a step of injecting the PCR reaction solution into the chamber; and a step of determining whether or not a complementary single-stranded DNA in the PCR reaction solution forms a hydrogen bond to a single-stranded DNA on each spot.

According to a third aspect of the present invention, a DNA testing system described below is provided. The DNA testing system comprises: a DNA testing chip; and a DNA testing chip control device. The DNA testing chip comprises a chamber into which a PCR reaction solution is injected, and a sensor. The chamber comprises a region where a plurality of spots on each of which a single-stranded DNA forms a solid phase are aligned. The plurality of aligned spots are formed in such a way that combinations of an associated genetic locus and an associated number of repeats are different from one another. The single-stranded DNA comprises an STR sequence having a genetic locus and a number of repeats associated with each of the spots. The sensor is used for determining whether or not a complementary single-stranded DNA in the PCR reaction solution forms a hydrogen bond to a single-stranded DNA on each spot. The DNA testing chip control device performs processing of injecting the PCR reaction solution into the chamber, and processing of determining whether or not a complementary single-stranded DNA in the PCR reaction solution forms a hydrogen bond to a single-stranded DNA on each spot.

According to a fourth aspect of the present invention, a DNA testing chip control device described below is provided. The DNA testing chip control device performs a DNA test by employing a DNA testing chip. The DNA testing chip comprises a chamber into which a PCR reaction solution is injected, and a sensor. The chamber comprises a region where a plurality of spots on each of which a single-stranded DNA forms a solid phase are aligned. The plurality of aligned spots are formed in such a way that combinations of an associated genetic locus and an associated number of repeats are different from one another. The single-stranded DNA comprises an STR sequence having a genetic locus and a number of repeats associated with each of the spots. The sensor is used for determining whether or not a complementary single-stranded DNA in the PCR reaction solution forms a hydrogen bond to a single-stranded DNA on each spot. The DNA testing chip control device performs processing of injecting the PCR reaction solution into the chamber, and processing of determining whether or not a complementary single-stranded DNA in the PCR reaction solution forms a hydrogen bond to a single-stranded DNA on each spot.

### Advantageous Effects of Invention

According to respective aspects of the present invention, a DNA testing chip, a DNA testing method, a DNA testing system, and a DNA testing control device, which contribute to performing a DNA test with a simplified processing process and a simplified processing mechanism are provided.

### BRIEF DESCRIPTION OF DRAWINGS

FIG. 1 is a diagram for describing an outline of a DNA testing chip 100 according to an example embodiment.

FIG. 2A is a diagram for describing an outline of the DNA testing chip 100 according to the example embodiment before a PCR reaction solution is injected.

FIG. 2B is a diagram for describing an outline of the DNA testing chip 100 according to the example embodiment when a PCR reaction solution is injected.

FIG. 2C is a diagram for describing an outline of the DNA testing chip 100 according to the example embodiment after washing.

FIG. 3 is a diagram illustrating a specific example of the DNA testing chip 100.

FIG. 4A is a diagram for describing a flow path control mechanism on a DNA preparation chip 101.

FIG. 4B is a diagram for describing a state of the flow path control mechanism on the DNA preparation chip 101.

FIG. 4C is a diagram for describing another state of the flow path control mechanism on the DNA preparation chip 101.

FIG. 5 is a diagram illustrating an example of a configuration of the DNA testing chip 100.

FIG. 6 is a diagram illustrating an example of a configuration of a swab receiving portion 116.

FIG. 7A is a diagram illustrating an external appearance of an example of a configuration of a testing chip 102.

FIG. 7B is a diagram illustrating details of an example of a configuration of the testing chip 102.

FIG. 7C is a diagram illustrating a cross section of an example of a configuration of the testing chip 102.

FIG. 8 is a diagram illustrating an example of a DNA testing chip control device 200.

FIG. 9 is a block diagram illustrating a configuration of a controller 223.

FIG. 10 is a diagram illustrating an example of information to be stored in an RAM 253.

FIG. 11 is a diagram illustrating an example of information to be displayed on a display unit 222.

FIG. 12 is a diagram illustrating an example of information to be stored in an ROM 252.

FIG. 13A is a diagram for describing a sequence of a free single-stranded DNA 162.

FIG. 13B is a diagram for describing a sequence of a solid-phase single-stranded DNA 161.

FIG. 14 is a flowchart describing a flow of processing by the DNA testing chip control device 200.

FIG. 15 is a diagram illustrating an example of a testing chip 102 according to a second example embodiment.

FIG. 16A is a diagram for describing detection principles of an SPR sensor.

FIG. 16B is another diagram for describing detection principles of the SPR sensor.

FIG. 17A is a first diagram for describing detection principles of an FRET sensor.

FIG. 17B is a second diagram for describing detection principles of the FRET sensor.

FIG. 17C is a third diagram for describing detection principles of the FRET sensor.

FIG. 18A is a fourth diagram for describing detection principles of the FRET sensor.

FIG. 18B is a fifth diagram for describing detection principles of the FRET sensor.

### EXAMPLE EMBODIMENT

### First Example Embodiment

Preferred example embodiments of the present invention are described in detail with reference to the drawings. Note that reference numbers in the drawings provided in the following description are provided to respective elements for convenience as an example for aiding understanding, and are not intended to limit the present invention to illustrated embodiments.

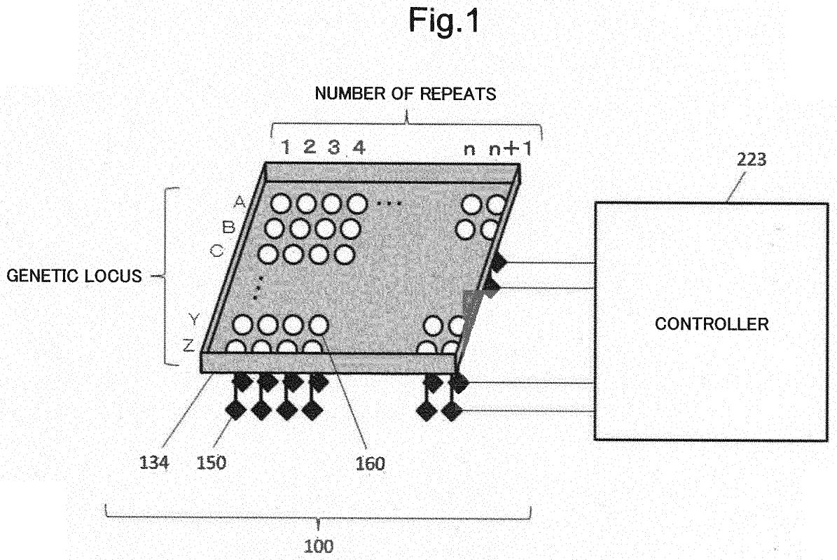

First of all, an outline of a DNA testing chip according to an example embodiment is described. As illustrated in FIG. 1, a DNA testing chip 100 comprises a detection chamber tank 134 into which a PCR reaction solution is injected, and a sensor 150. The detection chamber tank 134 includes a region where a plurality of spots 160 on each of which a single-stranded DNA forms a solid phase are aligned. As illustrated in FIG. 1, for example, the spots 160 are provided in such a way that combinations of an associated genetic locus and an associated number of repeats are different from one another. Note that, in the following, a spot 160 associated with a genetic locus A and a number of repeats n is described as a “spot (A, n)” or simply as “(A, n)”.

A solid-phase single-stranded DNA 161 which forms a solid phase on a spot 160 comprises an STR sequence having a genetic locus and a number of repeats associated with each of the spots 160. Note that, in the following, the solid-phase single-stranded DNA 161 is described as an “SP-ssDNA” (solid-phase single strand DNA), and SP-ssDNA associated with a genetic locus A and a number of repeats n is described as “SP-ssDNA (A, n)”.

The sensor 150 is connected to a controller 223 in a DNA testing chip control device. The controller 223 determines whether or not a complementary single-stranded DNA in a PCR reaction solution forms a hydrogen bond to a solid-phase single-stranded DNA 161 on respective spots 160 via the sensor 150. Note that a free single-stranded DNA 162 present in a free state in a PCR reaction solution is described as “free-ssDNA”, and free-ssDNA associated with a genetic locus A and a number of repeats n is described as “free-ssDNA (A, n)”.

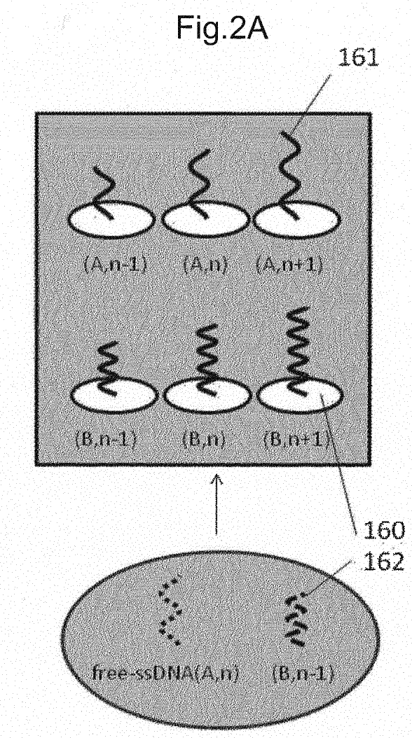

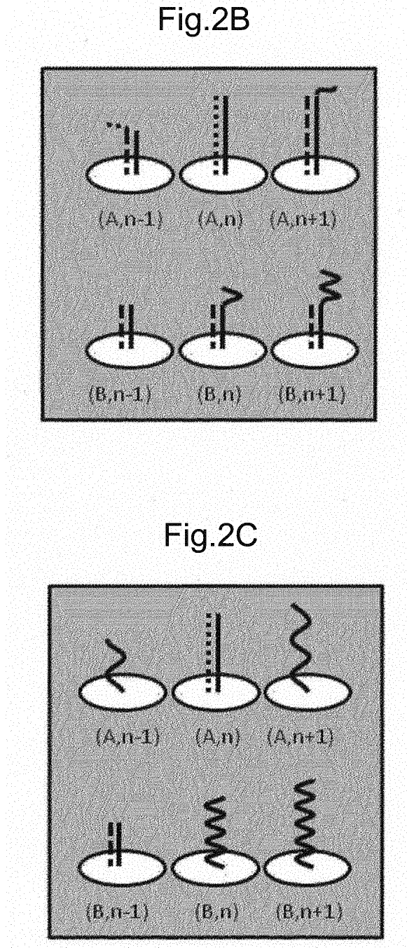

Next, a DNA test using the DNA testing chip 100 is conceptually described with reference to FIG. 2. As illustrated in FIG. 2A, spots (A, n−1), (A, n), and (A, n+1) associated with a genetic locus A, and spots (B, n−1), (B, n), and (B, n+1) associated with a genetic locus B are aligned on a bottom surface of the detection chamber tank 134. A PCR reaction solution is prepared by performing PCR for the genetic locus A and the genetic locus B all at once and by further performing denaturation. The PCR reaction solution includes free-ssDNA (A, n) and (B, n−1).

When the above-described PCR reaction solution is injected into the detection chamber tank 134, as illustrated in FIG. 2B, free-ssDNA (A, n) forms a hydrogen bond to SP-ssDNA (A, n−1), (A, n), and (A, n+1). At this occasion, since free-ssDNA (A, n) and SP-ssDNA (A, n) have same sequence lengths, a blunt double-stranded DNA is produced on the spot (A, n). Free-ssDNA (A, n) is short of a sequence corresponding to one repeat, as compared with SP-ssDNA (A, n+1), and is surplus of a sequence corresponding to one repeat, as compared with SP-ssDNA (A, n−1). Therefore, an overhung double-stranded DNA including a protruding terminus, or a double-stranded DNA of a bubble structure is formed on the spots (A, n−1) and (A, n+1). On the other hand, since free-ssDNA (A, n) and SP-ssDNA (B, n−1), (B, n), and (B, n+1) are not complementary to each other, a double-stranded DNA is not produced.

Likewise, free-ssDNA (B, n−1) forms a blunt double-stranded DNA on the spot (B, n−1), and forms an overhung double-stranded DNA or a double-stranded DNA of a bubble structure on the spots (B, n) and (B, n+1).

When the detection chamber tank 134 is washed in a state illustrated in FIG. 2B, a hydrogen bond in an overhung DNA and DNA of a bubble structure unbinds due to a single-stranded moiety thereof. Specifically, free-ssDNA (A, n) is dissociated from SP-ssDNA (A, n−1) and (A, n+1), and free-ssDNA (B, n−1) is dissociated from SP-ssDNA (B, n) and (B, n+1). Consequently, as illustrated in FIG. 2C, SP-ssDNA on respective spots 160 returns to a state of SP-ssDNA alone or turns to a state of a blunt double-stranded DNA.

In a state illustrated in FIG. 2C, the controller 223 determines whether or not a complementary free-ssDNA in a PCR reaction solution forms a hydrogen bond with SP-ssDNA on respective spots 160 (specifically, presence or absence of a double-stranded DNA) via the sensor 150. The sensor 150 is, for example, a quartz crystal microbalance (QCM) sensor, a surface plasmon resonance (SPR) sensor, or a fluorescence resonance energy transfer (FRET) sensor. At this occasion, the controller 223 acquires a positive determination result on the spots (A, n) and (B, n−1). In other words, it is found that free-ssDNA (A, n) and (B, n−1) are included in the PCR reaction solution.

In this way, by using the DNA testing chip 100, it is possible to perform a DNA test with a simplified processing process and a simplified processing mechanism.

Further, in a DNA test using the DNA testing chip 100, even when PCR for a plurality of genetic loci is performed all at once, it is possible to measure a number of repeats in an STR sequence in respective genetic loci. Therefore, it is possible to reduce labor in a DNA test such as sample dispensing.

Further, in the DNA testing chip 100, a number of repeats in an STR sequence is not measured based on a sequence length, but is measured based on sequence complementarity. Therefore, the DNA testing chip 100 does not require a constituent element (such as a capillary) for electrophoresis, and is advantageous in terms of cost reduction and downsizing.

### Second Example Embodiment

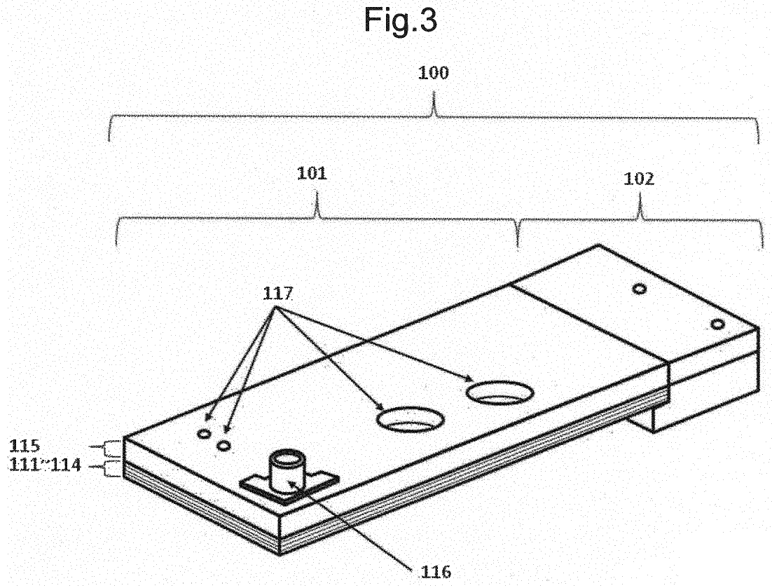

First of all, as a second example embodiment, an example of a DNA testing chip 100, and a DNA testing chip control device 200 for controlling the DNA testing chip 100 is described. As illustrated in FIG. 3, the DNA testing chip 100 is constituted by combining a DNA preparation chip 101 and a testing chip 102. The DNA preparation chip 101 is constituted by laminating elastic sheets 111 to 114, and a resin plate 115. A swab receiving portion 116 is mounted on the resin plate 115, and various types of control holes 117 passing through the resin plate 115 are formed.

The elastic sheets 111 to 114 have heat resistance and acid/alkali resistance, and contain silicon rubber and the like having elasticity as a main material. It is desirable that the resin plate 115 is hard to such an extent that extending the elastic sheets 111 to 114 is controllable. A part of the elastic sheets 111 to 114 is non-adhesive. A flow path 120, a liquid tank 121, a valve mechanism 123 and the like to be described later are formed by a non-adhesive portion. Note that, in the following drawings, a non-adhesive portion is indicated by a broken line.

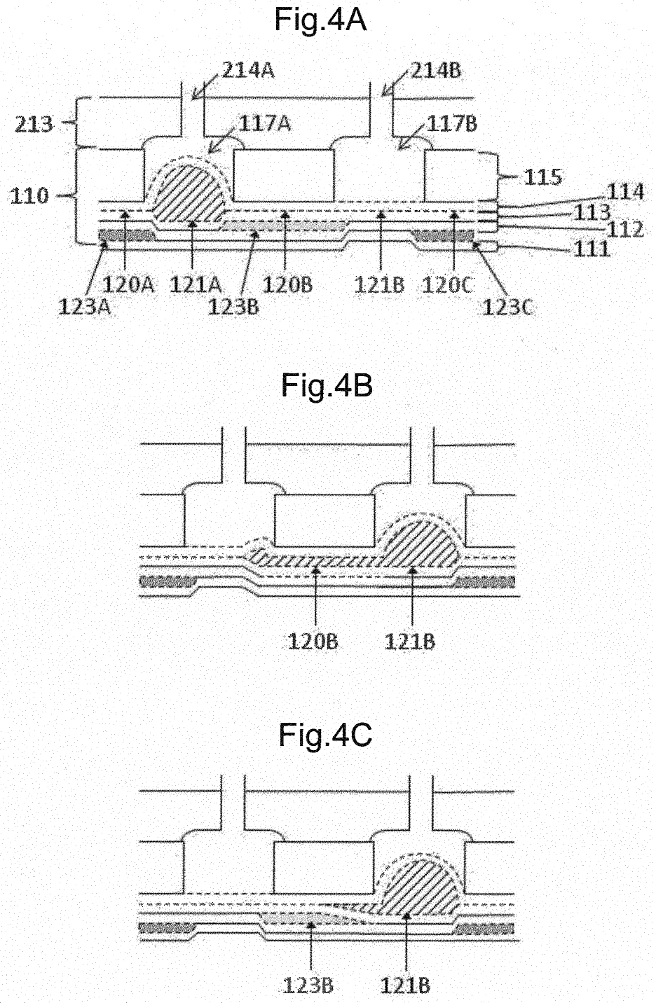

Herein, a basic structure of the DNA preparation chip 101, and an example of a flow path control mechanism are described using FIG. 4A, FIG. 4B, and FIG. 4C. As illustrated in FIG. 4A, the DNA preparation chip 101 is disposed in such a way that a lid 213 of the DNA testing chip control device 200 covers the resin plate 115. A non-adhesive portion is formed between the elastic sheet 113 and the elastic sheet 114 of the DNA preparation chip 101, and a portion serving as flow paths 120A to 120C and liquid tanks 121A and 121B is formed. A portion of the resin plate 115 associated with the liquid tanks 121A and 121B is a through portion, and serves as control holes 117A and 117B. A pressurizing medium (such as air) is taken in and out through pressurizing holes 214A and 214B formed in the lid 213 of the DNA testing chip control device 200.

Further, a portion serving as valve mechanisms 123A and 123C is formed between the elastic sheet 111 and the elastic sheet 112 of the DNA preparation chip 101. The valve mechanisms 123A and 123C are associated with the flow paths 120A and 120C, respectively. A pressurizing medium is taken in and out through the control holes 117 (not illustrated) passing through the elastic sheets 112 to 114, and through the pressurizing holes 214 (not illustrated) formed in the lid 213. Further, a portion serving as the valve mechanism 123B is formed between the elastic sheet 112 and the elastic sheet 113. A valve mechanism 123B is associated with the flow path 120B. A pressurizing medium is taken in and out through the control holes 117 (not illustrated) passing through the resin plate 115, and the elastic sheets 113 and 114, and through the pressurizing holes 214 (not illustrated) formed in the lid 213.

FIG. 4A illustrates a state that liquid is injected into the liquid tank 121A. At this occasion, a pressurizing medium is injected into the valve mechanisms 123A to 123C. The elastic sheet 113 is pushed up by expansion of the valve mechanisms 123A to 123C, and the flow paths 120A to 120C are closed.

When the flow path 120B is opened by releasing a pressurizing medium in the valve mechanism 123B, and the pressurizing medium is injected through the control hole 117A from a state illustrated in FIG. 4A, as illustrated in FIG. 4B, liquid within the liquid tank 121A reaches the liquid tank 121B through the flow path 120B. Specifically, liquid within the liquid tank 121A pushes down the elastic sheet 113, transfers downstream while forming the flow path 120B, pushes up the elastic sheet 114, forms the liquid tank 121B, and stays within the liquid tank 121B.

Thereafter, when a pressurizing medium is injected into the valve mechanism 123B from upstream side (specifically, from a side of the liquid tank 121A), as illustrated in FIG. 4C, liquid within the flow path 120B is squeezed out toward the liquid tank 121B. In this way, flow path control and liquid transport are performed on the DNA preparation chip 101.

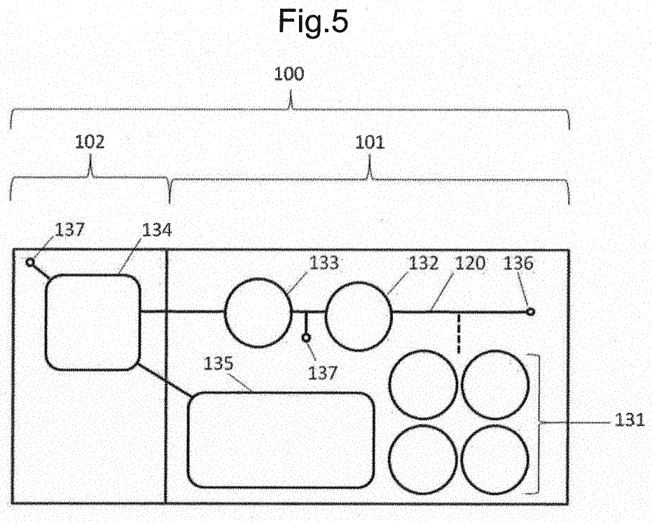

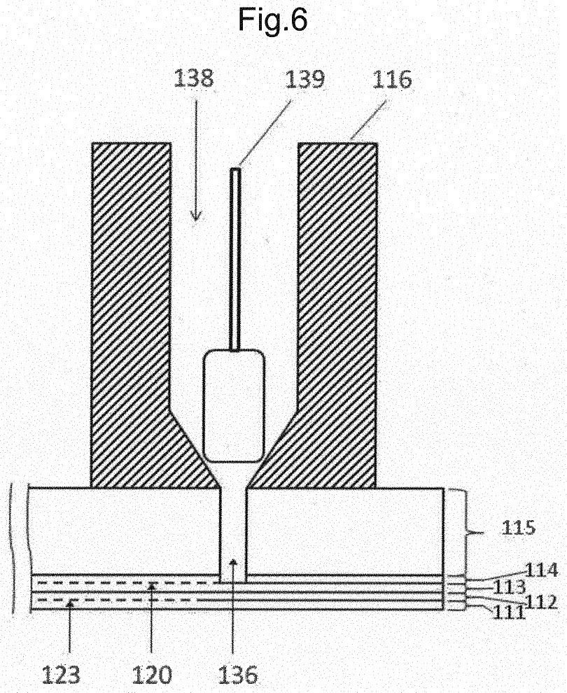

FIG. 5 is a diagram illustrating a layout of the flow path 120, the liquid tank 121, and the like on the DNA testing chip 100. As illustrated in FIG. 5, a buffer/reagent tank 131, a DNA extraction tank 132, a PCR tank 133, a detection chamber tank 134, and a washing buffer tank 135 as liquid tanks are formed on the DNA testing chip 100. Further, a sample injection hole 136 and a liquid discharge hole 137 are formed. Respective constituent elements are connected via the flow path 120. Note that a part of a configuration is omitted in order to simplify the drawing of FIG. 5.

A cell lysis buffer, a beads washing buffer, a DNA elution buffer, and the like are injected in advance in the buffer/reagent tank 131. The cell lysis buffer is an alkali lysis buffer for dissolving cells, for example. The beads washing buffer is a buffer for washing magnetic beads. The DNA elution buffer is a buffer for eluting DNA from magnetic beads. Note that the DNA elution buffer also includes a reagent for PCR (such as polymerase).

The buffer/reagent tank 131 is connected to a cell lysis tank 138 being an inner space of the swab receiving portion 116 via the flow path 120 and the sample injection hole 136 (see FIG. 6). Further, the buffer/reagent tank 131 is also connected to the DNA extraction tank 132 and the PCR tank 133 via the flow path 120.

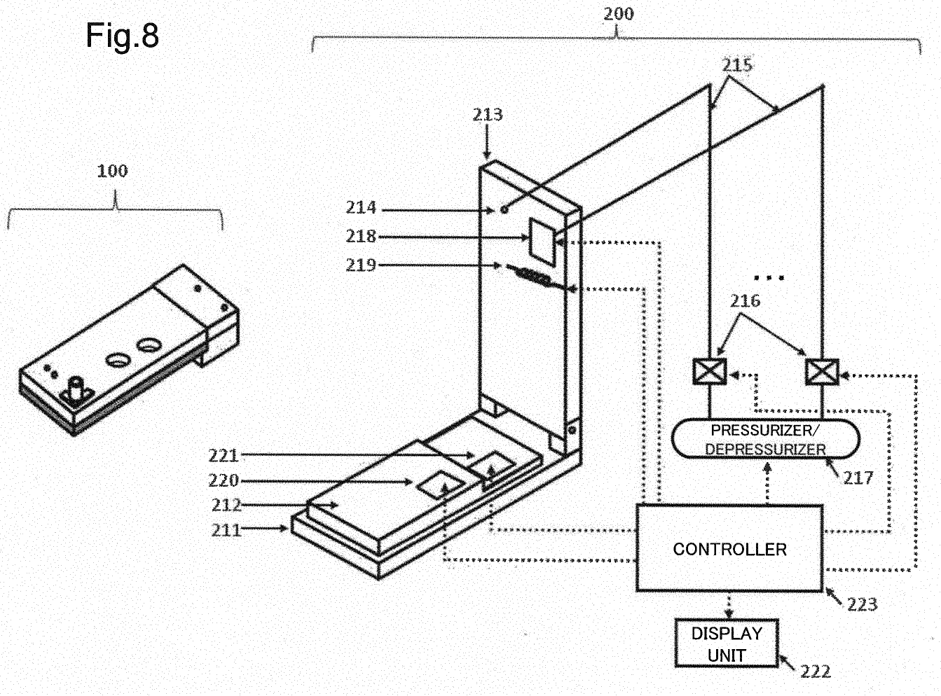

Specifically, as illustrated in FIG. 6, the cell lysis tank 138 is formed in an inner hollow portion of the tubular swab receiving portion 116, and is connected to the flow path 120 via the sample injection hole 136 as a lower opening portion. A swab 139 having cells of a subject attached thereto is placed in the cell lysis tank 138 through an upper opening portion, and when the lid 213 of the DNA testing chip control device 200 is closed, the swab receiving portion 116 is connected to a cell lysis unit 218 of the DNA testing chip control device 200 (see FIG. 8). At this occasion, the cell lysis tank 138 functions in a similar manner to the liquid tank 121A illustrated in FIG. 4A, except that liquid within the cell lysis tank 138 is directly squeezed out by a pressurizing medium.

Referring back to description of FIG. 5, the DNA extraction tank 132 is a liquid tank in which DNA extraction processing is performed. Specifically, magnetic beads (silica) are packed in advance inside the DNA extraction tank 132. DNA in a sample solution (specifically, a cell lysis buffer in which cells of a subject are dissolved) is adsorbed to magnetic beads. DNA extraction processing in the DNA extraction tank 132 is performed via a DNA extraction unit 219 of the DNA testing chip control device 200.

The PCR tank 133 is a liquid tank in which PCR is performed. Specifically, a plurality of sets of primers for amplifying an STR sequence are packed in advance inside the PCR tank 133. PCR for a plurality of genetic loci is performed all at once. PCR in the PCR tank 133 is performed via a PCR unit 220 of the DNA testing chip control device 200.

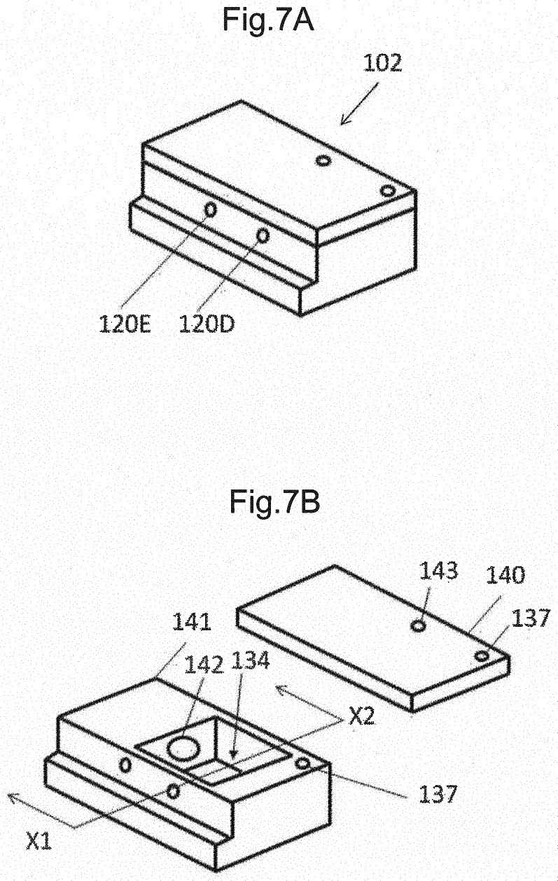

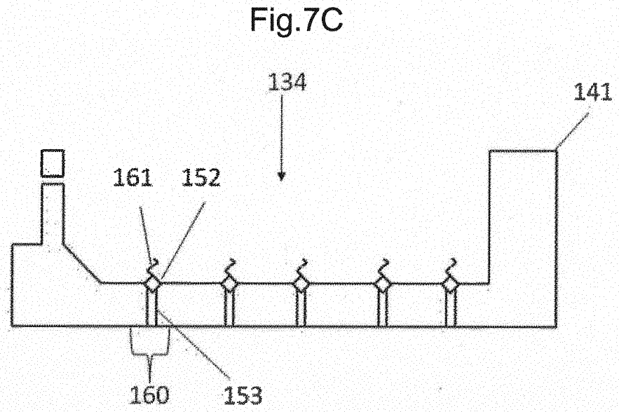

The detection chamber tank 134 is formed on the testing chip 102. Specifically, the testing chip 102 as a single member has an external appearance as illustrated in FIG. 7A, and is constituted by combining a resin lid portion 140 and a body portion 141, as illustrated in FIG. 7B. The detection chamber tank 134 is formed in the body portion 141, is connected to the PCR tank 133 of the DNA preparation chip 101 via a flow path 120D, and is connected to the washing buffer tank 135 via a flow path 120E. Further, a heater 142 for heating liquid within the detection chamber tank 134 is provided in the body portion 141.

Further, the liquid discharge hole 137 is formed in the lid portion 140 and the body portion 141. Liquid within the detection chamber tank 134 is discharged outside the testing chip 102 via the liquid discharge hole 137. A vent hole 143 is also formed in the lid portion 140. A positive pressure and a negative pressure generated within the detection chamber tank 134 are released when air is taken in and out through the vent hole 143.

FIG. 7C is a cross-sectional view of the body portion 141 taken along a line X1-X2 illustrated in FIG. 7B. As illustrated in FIG. 7C, crystal oscillators 152 on each of which SP-ssDNA (a solid-phase single-stranded DNA 161) forms a solid phase is aligned on a bottom surface of the detection chamber tank 134. The respective crystal oscillators 152 are connected to the controller 223 of the DNA testing chip control device 200 via input-output terminals 153 provided on the body portion 141.

Note that a spot 160 in FIG. 1 indicates a region where one crystal oscillator 152 is disposed in FIG. 7C. Further, the sensor 150 in FIG. 1 corresponds to a quartz crystal microbalance (QCM) sensor in FIG. 7C, and includes the crystal oscillators 152 and the input-output terminals 153. Note that an expression in disclosure of the present application i.e. a spot 160 on which a solid-phase single-stranded DNA 161 (SP-ssDNA) forms a solid phase is interpreted in FIG. 7C as a crystal oscillator 152 on which a solid-phase single-stranded DNA 161 (SP-ssDNA) forms a solid phase. In other words, respective crystal oscillators 152 are disposed in such a way that combinations of an associated genetic locus and an associated number of repeats are different from one another, and SP-ssDNA includes an STR sequence having a genetic locus and a number of repeats associated with respective crystal oscillators 152. A sequence of SP-ssDNA will be described later in detail.

Referring back to description of FIG. 5, a washing buffer for washing the detection chamber tank 134 is injected in advance in the washing buffer tank 135. A washing buffer is prepared in such a way as to secure a stringent condition (specifically, a condition that a hydrogen bond in an overhung DNA and DNA of a bubble structure unbinds). A washing buffer may be a plurality of types of washing buffers. In this case, a plurality of washing buffer tanks 135 are provided, and respective washing buffers are individually stored.

FIG. 8 is a diagram illustrating an example of the DNA testing chip control device 200. As illustrated in FIG. 8, in the DNA testing chip control device 200, a table 212 is disposed on a base 211, and the lid 213 openable via a hinge is provided. The DNA testing chip 100 is placed at a predetermined position on the table 212 by fitting a pin provided on the table 212 in a pinhole formed in the DNA testing chip 100, for example.

The plurality of pressurizing holes 214 are formed in the lid 213. The pressurizing holes 214 are respectively formed for the liquid tank 121, the valve mechanism 123, and the liquid discharge hole 137 in the DNA testing chip 100. In FIG. 8, the pressurizing holes 214 are omitted except for a part thereof for clarifying the drawing. A region of the lid 213 associated with the pressurizing holes 214 is a through portion, and the pressurizing holes 214 are connected to solenoid valves 216 via tubes 215. Further, the pressurizing holes 214 are connected to the control holes 117 and the liquid discharge hole 137 in the DNA testing chip 100 when the lid 213 is closed. Note that the pressurizing holes 214 and the control holes 117 may preferably be in firm contact with a sealing mechanism such as an O-ring interposed therebetween.

The solenoid valves 216 are connected to a pressurizer/depressurizer 217. A pressurizing medium such as compressed air is packed in the pressurizer/depressurizer 217. A pressurizing medium is taken in and out through the control holes 117 in the DNA testing chip 100 via the solenoid valves 216 and the pressurizing holes 214 (see FIG. 4A, FIG. 4B, and FIG. 4C). Further, the pressurizer/depressurizer 217 also functions as a pressure reducer. Liquid is discharged from the DNA testing chip 100 by sucking the liquid via the liquid discharge hole 137. Note that an inner pressure of the pressurizer/depressurizer 217 is controlled as to maintain a predetermined pressure by an unillustrated pressure sensor, an unillustrated pump, and the like.

Further, the cell lysis unit 218 and the DNA extraction unit 219 are also provided on the lid 213. The cell lysis unit 218 is connected to the swab receiving portion 116 on the DNA testing chip 100. Specifically, the cell lysis unit 218 includes a heater for heating a cell lysis buffer within the cell lysis tank 138. The DNA extraction unit 219 is an electromagnet, a neodymium magnet, or the like, for example, and holds or releases magnetic beads packed in the DNA extraction tank 132.

The PCR unit 220 and a detection unit 221 are provided on the table 212. The PCR unit 220 includes a temperature sensor, a heat transfer member, a Peltier element (a thermoelectric element), a heat radiating plate, and the like; and controls a temperature of the PCR tank 133 on the DNA testing chip 100. The detection unit 221 is an interface in contact with the heater 142 and the input-output terminals 153 on the testing chip 102.

The DNA testing chip control device 200 further includes a display unit 222 and the controller 223. The display unit 222 is a display, a monitor, and the like, for example. The controller 223 is a computer for controlling constituent elements of the DNA testing chip control device 200.

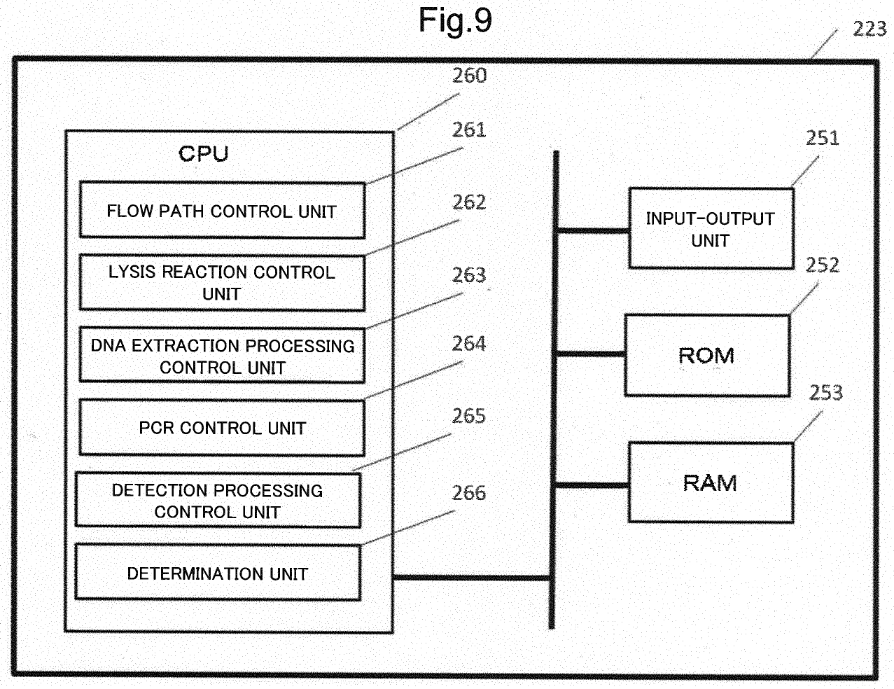

FIG. 9 is a block diagram illustrating a configuration of the controller 223. As illustrated in FIG. 9, the controller 223 is configured by connecting an input-output unit 251, a read only memory (ROM) 252, a random access memory (RAM) 253, and a central processing unit (CPU) 260 via a bus and the like.

The input-output unit 251 is an interface for connecting respective constituent elements of the DNA testing chip control device 200, and respective constituent elements of the controller 223. Further, the input-output unit 251 is connected to an operation device such as a keyboard, receives an input by a user, and transmits the input to the CPU 260.

The ROM 252 is a storage unit in which a program for controlling respective constituent elements of the DNA testing chip control device 200 is stored. The RAM 253 is a storage unit for use when a program stored in the ROM 252 is executed.

The CPU 260 executes a program stored in the ROM 252 by using the RAM 253 and the like. Various processing modules i.e. a flow path control unit 261, a lysis reaction control unit 262, a DNA extraction processing control unit 263, a PCR control unit 264, a detection processing control unit 265, and a determination unit 266 are implemented by the CPU 260 executing a program.

The flow path control unit 261 controls the solenoid valve 216 and the pressurizer/depressurizer 217 to perform flow path control and liquid transport on the DNA preparation chip 101, and discharge of liquid from the DNA preparation chip 101. Regarding flow path control and liquid transport on the DNA preparation chip 101, see FIG. 4A, FIG. 4B, FIG. 4C, and the like.

The lysis reaction control unit 262 controls the cell lysis unit 218 to perform a lysis reaction of cells of a subject. Specifically, when the DNA testing chip 100 is set on the DNA testing chip control device 200, the lysis reaction control unit 262 receives an input on a processing start instruction by a user. The lysis reaction control unit 262 instructs the flow path control unit 261 to transfer a cell lysis buffer from the buffer/reagent tank 131 to the cell lysis tank 138. Further, the lysis reaction control unit 262 carries out lysis reaction by controlling the cell lysis unit 218 in such a way as to heat the cell lysis buffer within the cell lysis tank 138 via a power supply unit (not illustrated).

The DNA extraction processing control unit 263 extracts DNA from a sample solution by controlling the DNA extraction unit 219. Specifically, the DNA extraction processing control unit 263 instructs the flow path control unit 261 to transfer a sample solution (specifically, a cell lysis buffer into which cells of a subject are dissolved) from the cell lysis tank 138 to the DNA extraction tank 132. Further, the DNA extraction processing control unit 263 instructs the flow path control unit 261 to transfer and discharge a beads washing buffer, while controlling holding or releasing of the magnetic beads by the DNA extraction unit 219, via a power supply unit (not illustrated). At this occasion, DNA in the sample solution is adsorbed to the magnetic beads.

The PCR control unit 264 performs PCR by controlling the PCR unit 220. Specifically, the PCR control unit 264 instructs the flow path control unit 261 to transfer a DNA elution buffer from the buffer/reagent tank 131 to the DNA extraction tank 132. At this occasion, DNA adsorbed to magnetic beads is eluted in the DNA elution buffer. Further, the PCR control unit 264 instructs the flow path control unit 261 to transfer the DNA elution buffer from the DNA extraction tank 132 to the PCR tank 133, and subsequently, performs PCR by controlling the PCR unit 220. Note that PCR is finished in a state that amplified DNA is denatured to a single-stranded DNA (e.g. a state that amplified DNA is retained at a temperature of 98° C.), for example.

The detection processing control unit 265 detects binding of a complementary free single-stranded DNA 162 to a solid-phase single-stranded DNA 161 which forms a solid phase. Specifically, the detection processing control unit 265 instructs the flow path control unit 261 to transfer a PCR reaction solution from the PCR tank 133 to the detection chamber tank 134. At this occasion, a free single-stranded DNA 162 in a PCR reaction solution binds to a solid-phase single-stranded DNA 161 which forms a solid phase on a crystal oscillator 152 (see FIG. 2B). Further, the detection processing control unit 265 instructs the flow path control unit 261 to transfer and discharge a washing buffer, while controlling the heater 142 of the testing chip 102 via the detection unit 221. Specifically, in the detection chamber tank 134, washing of a crystal oscillator 152 is performed, and DNA returns to a state of a single-stranded DNA which forms a solid phase on a crystal oscillator 152, or turns to a state of a double-stranded DNA (see FIG. 2C).

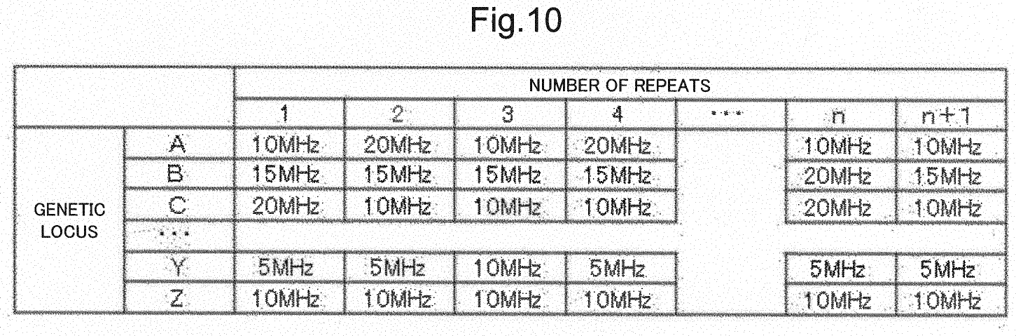

Herein, the detection processing control unit 265 oscillates respective crystal oscillators 152 by controlling a power supply unit (not illustrated) and applying alternate-current voltage to the respective crystal oscillators 152. Further, the detection processing control unit 265 counts a frequency of the oscillation, and stores the counted frequency in the RAM 253 in association with a genetic locus and a number of repeats. Information illustrated in FIG. 10 is stored in the RAM 253, for example.

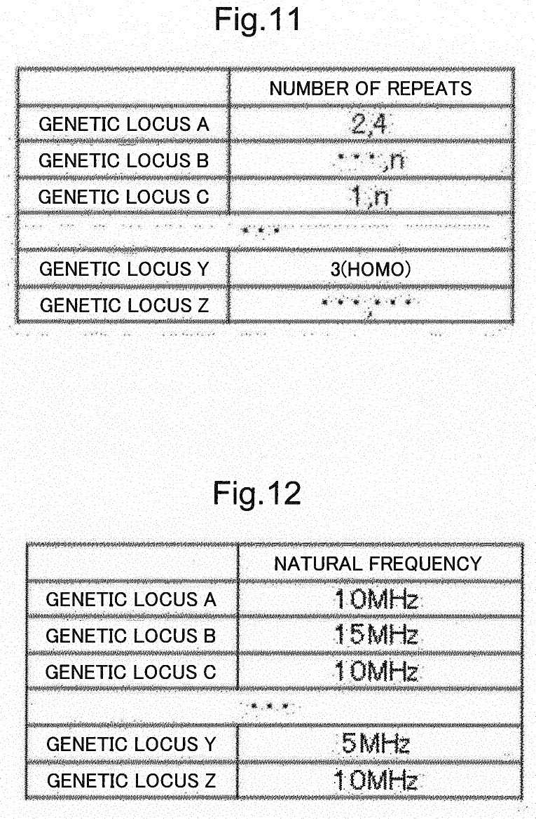

The determination unit 266 determines whether or not a free single-stranded DNA 162 in a PCR reaction solution forms a hydrogen bond with a solid-phase single-stranded DNA 161 on respective spots 160. Specifically, the determination unit 266 reads a frequency associated with a genetic locus and a number of repeats from the RAM 253, and determines a number of repeats at which a frequency is high in respective genetic loci. For example, in FIG. 10, it is determined that frequencies at the number of repeats 2 and the number of repeats 4 are higher than a frequency at the other numbers of repeats regarding a genetic locus A. Further, the determination unit 266 outputs, to the display unit 222, a name of a genetic locus, and the determined number of repeats in association with each other. In other words, a number of repeats in an STR sequence of a subject is displayed on the display unit 222 in association with a name of a genetic locus. FIG. 11 is an example of information to be displayed on the display unit 222.

Note that, as illustrated in FIG. 10 and FIG. 11, since humans are diploid organisms, there are a case where a two numbers of repeats at which a frequency is high are determined (in other words, hetero-DNA), and a case where a single number of repeats at which a frequency is high is determined (in other words, homo-DNA). When a single number of repeats is determined, information indicating “homo-DNA” as illustrated by a genetic locus Y in FIG. 11 may be supplementarily provided.

Further, the determination unit 266 may compare a natural frequency of respective genetic loci measured in advance and stored in the ROM 252, and a frequency read from the RAM 253. For example, information illustrated in FIG. 12 is stored in advance in the ROM 252.

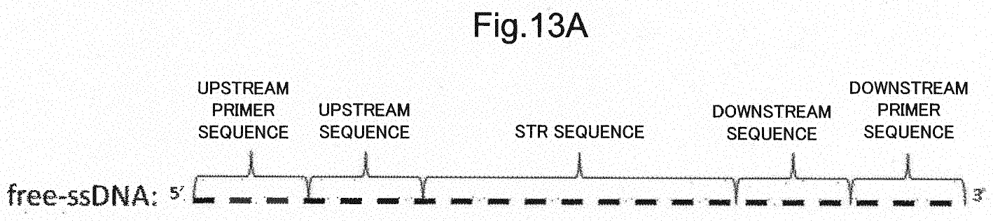

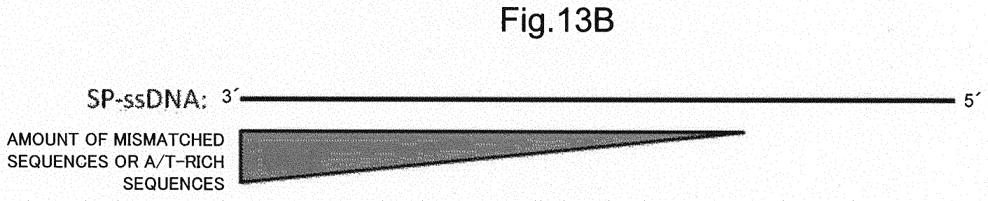

In the following, sequences of a solid-phase single-stranded DNA 161 and a free single-stranded DNA 162 are described. In the present example embodiment, a region including an STR sequence is amplified by PCR. Specifically, a primer of PCR is designed to anneal to an upstream portion and a downstream portion of a STR sequence. Therefore, as illustrated in FIG. 13A, a free single-stranded DNA 162 (free-ssDNA) present in a free state in a PCR reaction solution includes an upstream primer sequence, an upstream sequence, an STR sequence, a downstream sequence, and a downstream primer sequence. Herein, it is possible to design any sequence on a 5′ end of an upstream primer sequence and a 3′ end of a downstream primer sequence. On the other hand, a solid-phase single-stranded DNA 161 (SP-ssDNA) which forms a solid phase on a spot 160 is synthetic oligonucleotide, and it is possible to design any length and any sequence.

In the second example embodiment, when sequence lengths of free-ssDNA and SP-ssDNA are the same, a blunt double-stranded DNA is produced. Therefore, in SP-ssDNA, basically, a sequence complementary to free-ssDNA is designed. However, a mismatched sequence or an A/T-rich sequence may be incorporated in SP-ssDNA. For example, as illustrated in FIG. 13B, SP-ssDNA is designed in such a way that an amount of mismatched sequences or A/T-rich sequences increases from an upstream portion toward a downstream portion. Whereas an upstream portion of SP-ssDNA having a sequence as described above stably binds to free-ssDNA, a downstream portion thereof transiently binds to free-ssDNA (in other words, binding and unbinding are repeated). Consequently, since free-ssDNA and SP-ssDNA gradually bind from a downstream portion toward an upstream portion of SP-ssDNA, a blunt double-stranded DNA is produced. At this occasion, it is preferable to gradually lower a temperature of a buffer, or gradually reduce a salt concentration of a buffer.

Further, in order to uniquely unbind a hydrogen bond of an overhung double-stranded DNA and a double-stranded DNA of a bubble structure, while retaining a blunt double-stranded DNA, a chaotropic agent (e.g. urea and formamide) may be used. An overhung double-stranded DNA may have a Y-fork-shaped cleavage portion in an upstream portion or a downstream portion thereof, and a double-stranded DNA of a bubble structure may have a cleavage portion in an STR sequence. A chaotropic agent preferentially enters the cleavage portion and unstabilizes a double-stranded structure. Therefore, using a chaotropic agent may make it easy to cause dissociation of an overhung double-stranded DNA and a double-stranded DNA of a bubble structure, as compared with a blunt double-stranded DNA. Further, a DNA helicase which functions as to further open a cleavage portion of a double-stranded DNA may be used (e.g. “You et. al., The EMBO Journal, November 17 (2003), Volume 22, Issue 22: P. 6148-60.” is cited as a reference document).

Note that the sensor 150 may uniquely detect a spot 160 on which a blunt double-stranded DNA binds in a state that a blunt double-stranded DNA, an overhung double-stranded DNA, or a double-stranded DNA of a bubble structure is present on a spot 160 (see FIG. 2B). For example, a frequency when a blunt double-stranded DNA is present, and a frequency when an overhung double-stranded DNA or a double-stranded DNA of a bubble structure is present on respective spots 160 may be measured in advance. Further, it may be determined whether a blunt double-stranded DNA is present, or an overhung double-stranded DNA or a double-stranded DNA of a bubble structure is present on respective spots 160 by comparing with a frequency to be measured at implementation. At this occasion, for example, a base of a molecular weight larger than a normal base may be incorporated in a single-stranded moiety of an overhung double-stranded DNA by using a Klenow enzyme, or an intercalator that uniquely fits in a bubble structure moiety may be used. By using a Klenow enzyme or an intercalator, a weight of an overhung double-stranded DNA or a double-stranded DNA of a bubble structure is made heavier than a blunt double-stranded DNA. This causes a difference in frequency between crystal oscillators 152.

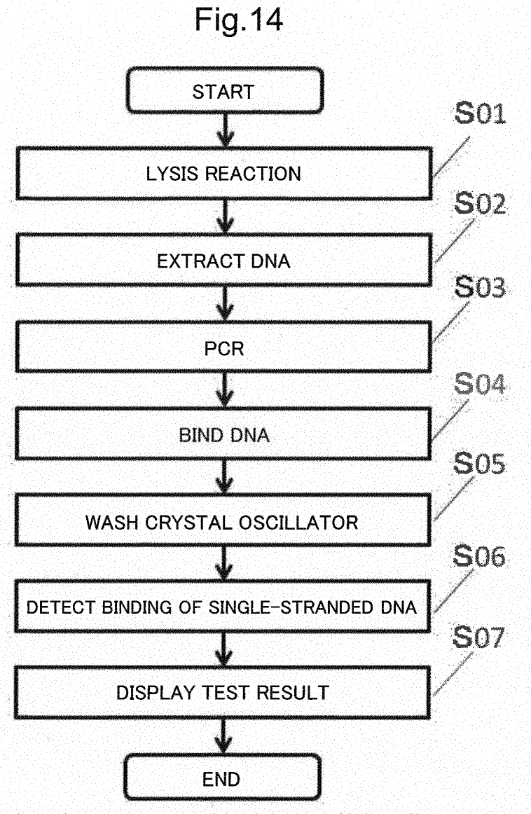

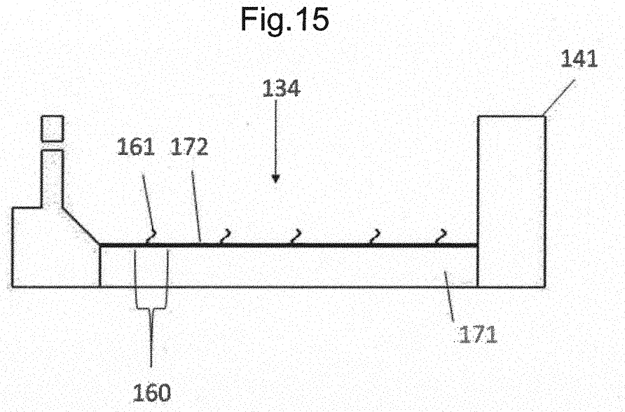

In the following, a flow of processing by the DNA testing chip control device 200 is described. As illustrated in FIG. 14, when an input on a processing start instruction by a user is received, the DNA testing chip control device 200 carries out a cell lysis reaction (Step S01), extracts DNA from a sample solution (Step S02), and performs PCR (Step S03). Subsequently, the DNA testing chip control device 200 causes free-ssDNA in a PCR reaction solution to bind to SP-ssDNA which forms a solid phase on a crystal oscillator 152 (Step S04), washes the crystal oscillator 152 (Step S05), and detects binding of the free-ssDNA to the SP-ssDNA (Step S06). Further, the DNA testing chip control device 200 displays a test result in which a name of a genetic locus and a number of repeats in an STR sequence are associated with each other (Step S07).

As described above, in the DNA testing chip 100 of the second example embodiment, PCR for a plurality of genetic loci is performed all at once, and a number of repeats in an STR sequence is measured, based on sequence complementarity. Therefore, it is possible to reduce labor in a DNA test such as sample dispensing, and it is advantageous in terms of cost reduction and downsizing.

### Third Example Embodiment

In a third example embodiment, a case where the sensor 150 in FIG. 1 is a surface plasmon resonance (SPR) sensor employing surface plasmon resonance is described. Note that, in the following, points different from the first example embodiment are described.

In a body portion 141 of a testing chip 102 according to the third example embodiment, a glass plate 171 on which a thin film of gold particles is formed by vapor deposition is disposed on a bottom surface of a detection chamber tank 134, and SP-ssDNA forms a solid phase on a gold particle film 172. Note that a spot 160 in FIG. 1 indicates a region where SP-ssDNA of a single type forms a solid phase.

A detection unit 221 of a DNA testing chip control device 200 further includes a light source for irradiating laser light to the glass plate 171, and a camera (light receiving unit) for receiving reflected light.

A detection processing control unit 265 of a controller 223 captures reflected light on respective spots 160 by controlling the detection unit 221. Further, the detection processing control unit 265 specifies a portion where luminance of reflected light is low, and stores, in an RAM 253, information relating to the low luminance portion (e.g. an angle of reflection) in association with a genetic locus and a number of repeats of respective spots 160.

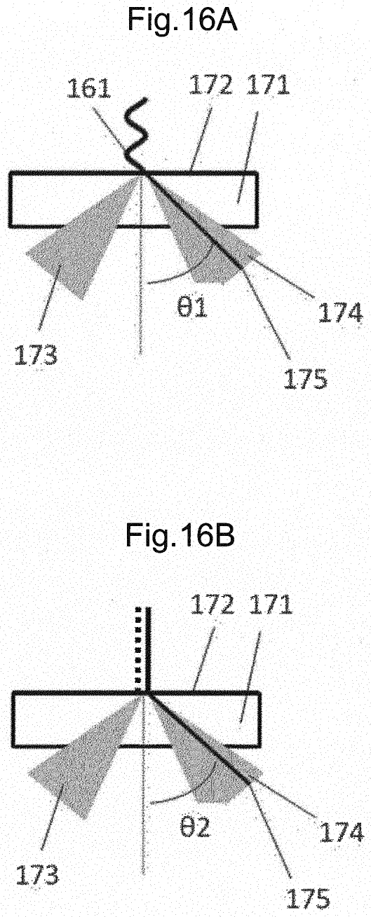

When description is made based on principles of an SPR sensor, as illustrated in FIG. 16A, incident light 173 is radiated to respective spots 160 by total reflection. Reflected light 174 caused by total reflection includes a low luminance portion 175 caused by SP-ssDNA which forms a solid phase on the gold particle film 172. The low luminance portion 175 is identified by an angle of reflection. For example, when SP-ssDNA is present alone, the low luminance portion 175 is indicated by an angle of reflection θ1. Further, as illustrated in FIG. 16B, the incident light 173 is also irradiated to a spot on which free-ssDNA binds to SP-ssDNA and a double-stranded DNA is produced. The low luminance portion 175 at this occasion is indicated by an angle of reflection θ2, which is different from an angle of reflection when a solid-phase single-stranded DNA 161 is present alone.

A determination unit 266 reads, from the RAM 253, an angle of reflection associated with a genetic locus and a number of repeats, and determines a number of repeats associated with a unique (specific) angle of reflection for respective genetic loci. Further, the determination unit 266 outputs, to a display unit 222, a name of a genetic locus and the determined number of repeats in association with each other.

In this way, it is possible to perform a DNA test even when a sensor is an SPR sensor employing surface plasmon resonance.

### Fourth Example Embodiment

In a fourth example embodiment, a case where the sensor 150 in FIG. 1 is a fluorescence resonance energy transfer (FRET) sensor employing fluorescence resonance energy transfer is described. Note that, in the following, points different from the second example embodiment are described.

SP-ssDNA which forms a solid phase on a spot 160 is synthesized in a state that a first fluorescent substance 181 binds to a 3′-terminus. Further, a primer packed in advance in a PCR tank 133 is synthesized in a state that a second fluorescent substance 182 (quencher) binds to a 5′-terminus in advance. In other words, free-ssDNA includes the second fluorescent substance 182 at a 5′-terminus.

A body portion 141 of a testing chip 102 according to the fourth example embodiment is made of, for example, a glass plate so that fluorescence emitted from the first fluorescent substance 181 can be observed from a side of a bottom surface of a detection chamber tank 134. SP-ssDNA directly forms a solid phase on a bottom surface of the detection chamber tank 134. Note that, in the fourth example embodiment, crystal oscillators 152 and input-output terminals 153 are not necessary. In the fourth example embodiment, a spot 160 in FIG. 1 indicates a region where SP-ssDNA of a single type forms a solid phase.

A detection unit 221 of a DNA testing chip control device 200 further includes a light source for irradiating excitation light, and a camera (light receiving unit) for receiving fluorescence.

A detection processing control unit 265 of a controller 223 captures fluorescence of respective spots 160 by controlling the detection unit 221. Further, the detection processing control unit 265 stores, in an RAM 253, a fluorescence luminance of respective spots 160 in association with a genetic locus and a number of repeats.

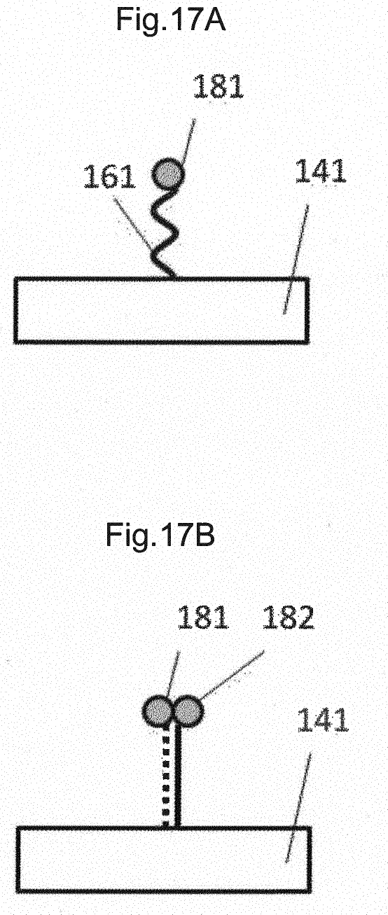

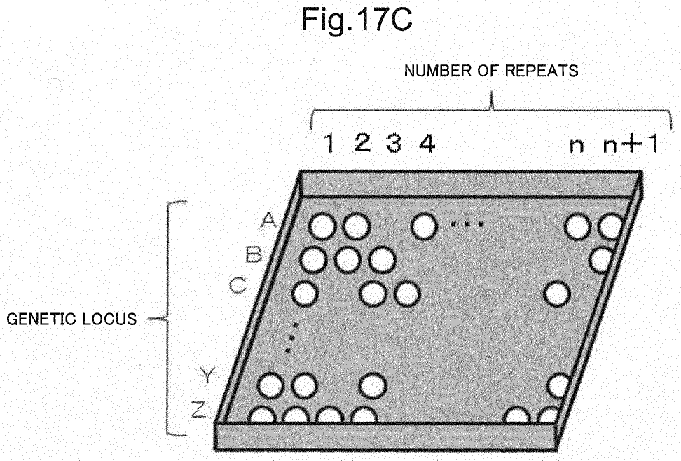

When description is made based on principles of a FRET sensor, as illustrated in FIG. 17A, when SP-ssDNA is present alone, the first fluorescent substance 181 is excited by excitation light irradiated from below the body portion 141 of the testing chip 102, and fluorescence of a first wavelength is emitted. On the other hand, as illustrated in FIG. 17B, in a state that free-ssDNA binds to SP-ssDNA and a blunt double-stranded DNA is produced, resonance energy transfer occurs since the first fluorescent substance 181 and the second fluorescent substance 182 (quencher) are proximate to each other. At this occasion, the first fluorescent substance 181 and the second fluorescent substance 182 emit fluorescence of a wavelength different from the first wavelength. In other words, as illustrated in FIG. 17C, when fluorescence of the first wavelength is captured, fluorescence is observed on a spot 160 on which SP-ssDNA is present alone, but fluorescence is not observed on a spot 160 on which SP-ssDNA turns to a blunt double-stranded DNA.

A determination unit 266 reads, from the RAM 253, a fluorescence luminance associated with a genetic locus and a number of repeats, and determines a number of repeats associated with a unique (specific) fluorescence luminance, or a fluorescence luminance of a value smaller than a predetermined value, for respective genetic loci. Further, the determination unit 266 outputs, to a display unit 222, a name of a genetic locus and the determined number of repeats in association with each other.

In this way, it is possible to perform a DNA test even when fluorescence resonance energy transfer is employed.

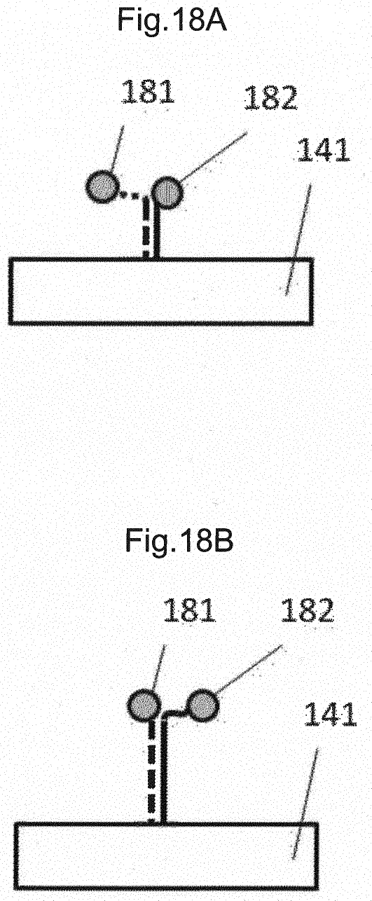

Note that, in the fourth example embodiment, it is possible to obtain a similar result even in a state that an overhung double-stranded DNA is present on a spot 160. Conceptually, even when a sequence length of SP-ssDNA is smaller than a sequence length of free-ssDNA as illustrated in FIG. 18A, or even in a case where a sequence length of SP-ssDNA is larger than a sequence length of free-ssDNA as illustrated in FIG. 18B, the first fluorescent substance 181 and the second fluorescent substance 182 (quencher) are not proximate to each other. Therefore, when fluorescence of the first wavelength is captured, resonance energy transfer does not occur on a spot 160 on which an overhung double-stranded DNA is present, and fluorescence of the first fluorescent substance 181 is observed. Note that, as illustrated in FIG. 13, it is possible to remove a bubble structure by designing SP-ssDNA in such a way that an amount of mismatched sequences or A/T-rich sequences increases from a downstream portion toward an upstream portion.

### Fifth Example Embodiment

In the following, various modifications are described as a fifth example embodiment. For example, as described in the first to fourth example embodiments, a sensor 150 is replaceable by various types of mechanisms. Specifically, a sensor 150 may include another mechanism, as long as determining whether or not complementary free-ssDNA in a PCR reaction solution forms a hydrogen bond to SP-ssDNA on respective spots 160 (specifically, presence or absence of a blunt double-stranded DNA).

Further, a spot 160 on which a blunt double-stranded DNA binds may be detected, based on a temperature at which a double-stranded DNA is produced or dissociated (e.g. a temperature at which a double-stranded DNA is produced with a probability of 50%, so-called a Tm value (melting temperature)). For example, a Tm value of a blunt double-stranded DNA is measured in advance regarding respective spots 160, and a database is prepared. Further, when implementation is performed, a frequency and an angle of reflection regarding respective spots are chronologically measured, while gradually increasing or decreasing a temperature of liquid within a detection chamber tank 134, and a change with respect to a temperature is expressed as a graph. From the graph, an actual Tm value regarding respective spots 160 is calculated, and it is determined whether or not a double-stranded DNA on a spot 160 is blunt by comparing the calculated Tm value with a Tm value in a database.

Note that it is conceived that a Tm value when an overhung structure or a bubble structure is produced is lower than a Tm value when a blunt double-stranded DNA is produced. For example, see SantaLucia J Jr and Hicks D, Annual Review of Biophysics and Biomolecular Structure (2004) Vol. 33: P. 415-440. Further, it is conceived that the larger a difference in number of repeats between SP-ssDNA and free-ssDNA is, the larger a drop range of the above-described Tm value is.

Further, a Tm value for free-ssDNA having all numbers of repeats including a case where an overhung structure or a bubble structure is produced and a case where a blunt double-stranded DNA is produced, regarding respective spots 160 may be collected in a database. Further, a Tm value for all combinations of free-ssDNA (including hetero-DNA and homo-DNA) regarding respective spots 160 may be collected in a database. Specifically, when free-ssDNA is hetero-DNA, not only a blunt double-stranded DNA but also a double-stranded DNA of an overhung structure or a bubble structure may be produced on a positive spot 160. Even in this case, it is possible to accurately determine whether or not a double-stranded DNA on a spot 160 is blunt. Further, determination as to whether or not a double-stranded DNA on a spot 160 is blunt may be made by comparing a change in frequency or angle of reflection with respect to a temperature from a graph, without comparing with a Tm value.

Further, in the first example embodiment, a DNA testing chip 100 constituted by combining a DNA preparation chip 101 and a testing chip 102 is described. Alternatively, a testing chip 102 may be used alone. Specifically, processing until preparation of a PCR reaction solution may be performed manually, and a DNA test may be performed by using a testing chip 102. In this case, particularly, labor such as sample dispensing in preparation of a PCR reaction solution is reduced.

Further, a DNA testing chip 100 is dispensable. Alternatively, a DNA preparation chip 101 and a testing chip 102 may be separably configured, and the testing chip 102 may be repeatedly used. In this case, a detection chamber tank 134 is returned to a state before use by being washed after each time of use, and a washing buffer tank 135 is refilled with a washing buffer.

Further, a configuration of a DNA preparation chip 101 may be modified in various ways. For example, DNA extraction processing in a DNA extraction tank 132 is not limited to processing in which magnetic beads are used, but may be processing in which a column is used.

Note that a part or an entirety of the above-described example embodiments may be described as the following supplementary notes, but are not limited to the following.

(Supplementary Note 1)

A DNA testing chip comprising:
a chamber into which a PCR reaction solution is injected; and
a sensor,
wherein the chamber comprises a region in which a plurality of spots, where a single-stranded DNA forms a solid phase, are aligned,
wherein each of the spots corresponds to deferent combination of genetic locus and number of repeats,
wherein each of the solid phase single-stranded DNA has a STR sequence with the genetic locus and the number of repeats which are corresponding to each of the spots, and
wherein the sensor is used for determining whether or not a complementary single-stranded DNA in the PCR reaction solution forms a hydrogen bond with the single-stranded DNA on each spot.

(Supplementary Note 2)

The DNA testing chip according to supplementary note 1, wherein the sensor is a quartz crystal microbalance (QCM) sensor employing a crystal oscillator, a surface plasmon resonance (SPR) sensor employing surface plasmon resonance, or a fluorescence resonance energy transfer (FRET) sensor employing fluorescence resonance energy transfer.

(Supplementary Note 3)

The DNA testing chip according to supplementary note 1 or 2, wherein a single-stranded DNA on a spot includes a primer sequence.

(Supplementary Note 4)

The DNA testing chip according to supplementary note 3, wherein the primer sequence includes a mismatched sequence.

(Supplementary Note 5)

The DNA testing chip according to any one of supplementary notes 1 to 4, further including a PCR reaction tank in which a PCR reaction is performed for STR sequences of a plurality of genetic loci.

(Supplementary Note 6)

The DNA testing chip according to supplementary note 5, further including a DNA extraction tank in which DNA is extracted from cells of a subject.

(Supplementary Note 7)

A DNA testing method employing a DNA testing chip comprising
preparing the DNA testing chip,
wherein the DNA testing chip comprises a chamber into which a PCR reaction solution is injected, and a sensor,
wherein the chamber comprises a region in which a plurality of spots, where a single-stranded DNA forms a solid phase, are aligned,
wherein each of the spots corresponds to deferent combination of genetic locus and number of repeats,
wherein each of the solid phase single-stranded DNA has an STR sequence with the genetic locus and the number of repeats which are corresponding to each of the spots, and
wherein the sensor is used for determining whether or not a complementary single-stranded DNA in the PCR reaction solution forms a hydrogen bond with the single-stranded DNA on each spot,
the DNA testing method further comprising:
   injecting the PCR reaction solution into the chamber; and
   determining whether or not a complementary single-stranded DNA in the PCR reaction solution forms a hydrogen bond to the single-stranded DNA on each spot.

(Supplementary Note 8)

A DNA testing system comprising:
a DNA testing chip; and
a DNA testing chip control device,
wherein the DNA testing chip comprises a chamber into which a PCR reaction solution is injected, and a sensor,
wherein the chamber comprises a region in which a plurality of spots, where a single-stranded DNA forms a solid phase, are aligned,
wherein each of the spots corresponds to deferent combination of genetic locus and number of repeats,
wherein each of the solid phase single-stranded DNA has an STR sequence with the genetic locus and the number of repeats which are corresponding to each of the spots,
wherein the sensor is used for determining whether or not a complementary single-stranded DNA in the PCR reaction solution forms a hydrogen bond with the single-stranded DNA on each spot, and
wherein the DNA testing chip control device performs
processing of injecting the PCR reaction solution into the chamber, and
processing of determining whether or not a complementary single-stranded DNA in the PCR reaction solution forms a hydrogen bond to a single-stranded DNA on each spot.

(Supplementary Note 9)

A DNA testing chip control device which performs a DNA test using a DNA testing chip,
wherein the DNA testing chip comprises a chamber into which a PCR reaction solution is injected, and a sensor,
wherein each of the solid phase single-stranded DNA has an STR sequence with the genetic locus and the number of repeats which are corresponding to each of the spots,
wherein the sensor is used for determining whether or not a complementary single-stranded DNA in the PCR reaction solution forms a hydrogen bond with the single-stranded DNA on each spot, and
the DNA testing chip control device performs
processing of injecting the PCR reaction solution into the chamber, and
processing of determining whether or not a complementary single-stranded DNA in the PCR reaction solution forms a hydrogen bond to a single-stranded DNA on each spot.

Note that it is assumed that disclosure of the above-described patent literature is incorporated in the present specification by reference. Modifications/adjustments of example embodiments and examples are available within the scope of all disclosure (including the claims) of the present invention, and further based on basic technical ideas thereof. Further, various combinations and selections of various disclosure elements (including respective elements of respective claims, respective elements of respective example embodiments and examples, respective elements of respective drawings, and the like) are available within the scope of the claims of the present invention. Specifically, it is needless to say that the present invention includes various modifications and alterations, which may be achieved by a person skilled in the art in accordance with all disclosure and technical ideas including the claims.

This application claims the priority based on Japanese Patent Application No. 2016-023542 filed on Feb. 10, 2016, the disclosure of which is incorporated herein in its entirety.

### REFERENCE SIGNS LIST

100 DNA testing chip
101 DNA preparation chip
102 Testing chip
111 to 114 Elastic sheet
115 Resin plate
116 Swab receiving portion
117 Control hole
120 Flow path
121 Liquid tank
123 Valve mechanism
131 Buffer/reagent tank
132 DNA extraction tank
133 PCR tank
134 Detection chamber tank
135 Washing buffer tank
136 Sample injection hole
137 Liquid discharge hole
138 Cell lysis tank
139 Swab
140 Lid portion
141 Body portion
142 Heater
143 Vent hole
150 Sensor
152 Crystal oscillator
153 Input-output terminal
160 Spot
161 Solid-phase single-stranded DNA
162 Free single-stranded DNA
171 Glass plate
172 Gold particle film
173 Incident light
174 Reflected light
175 Low luminance portion
181 First fluorescent substance
182 Second fluorescent substance
200 DNA testing chip control device
211 Base
212 Table
213 Lid
214 Pressurizing hole
215 Tube
216 Solenoid valve
217 Pressurizer/depressurizer
218 Cell lysis unit
219 DNA extraction unit
220 PCR unit
221 Detection unit
222 Display unit
223 Controller
251 Input-output unit
252 ROM
253 RAM
260 CPU
261 Flow path control unit
262 Lysis reaction control unit
263 DNA extraction processing control unit
264 PCR control unit
265 Detection processing control unit
266 Determination unit

What is claimed is:

1. A DNA testing chip comprising:
a chamber into which a PCR reaction solution is injected; and
a sensor,
wherein the chamber comprises a region in which a plurality of spots, where a single-stranded DNA forms a solid phase, are aligned,
wherein each of the spots corresponds to different combination of genetic locus and number of repeats,
wherein each of the solid phase single-stranded DNA has a STR sequence with the genetic locus and the number of repeats which are corresponding to each of the spots, and
wherein the sensor is used for determining whether or not a complementary single-stranded DNA in the PCR reaction solution forms a hydrogen bond with the single-stranded DNA on each spot.

2. The DNA testing chip according to claim 1,
wherein the sensor is a quartz crystal microbalance (QCM) sensor using a crystal oscillator, or a surface plasmon resonance (SPR) sensor using surface plasmon resonance, or a fluorescence resonance energy transfer (FRET) sensor using fluorescence resonance energy transfer.

3. The DNA testing chip according to claim 1,
wherein the single-stranded DNA on the spot comprises a primer sequence.

4. The DNA testing chip according to claim 3,
wherein the primer sequence comprises a mismatched sequence.

5. The DNA testing chip according to claim 1, further comprising
a PCR reaction tank in which a PCR reaction is performed for STR sequences of a plurality of genetic loci.

6. The DNA testing chip according to claim 5, further comprising a DNA extraction tank in which DNA is extracted from cells of a subject.

7. A DNA testing method employing a DNA testing chip comprising
preparing the DNA testing chip,
wherein the DNA testing chip comprises a chamber into which a PCR reaction solution is injected, and a sensor,
wherein the chamber comprises a region in which a plurality of spots, where a single-stranded DNA forms a solid phase, are aligned,
wherein each of the spots corresponds to different combination of genetic locus and number of repeats,
wherein each of the solid phase single-stranded DNA has an STR sequence with the genetic locus and the number of repeats which are corresponding to each of the spots, and
wherein the sensor is used for determining whether or not a complementary single-stranded DNA in the PCR reaction solution forms a hydrogen bond with the single-stranded DNA on each spot,
the DNA testing method further comprising:
injecting the PCR reaction solution into the chamber; and
determining whether or not a complementary single-stranded DNA in the PCR reaction solution forms a hydrogen bond to the single-stranded DNA on each spot.

8. A DNA testing system comprising:
a DNA testing chip; and
a DNA testing chip control device,
wherein the DNA testing chip comprises a chamber into which a PCR reaction solution is injected, and a sensor,
wherein the chamber comprises a region in which a plurality of spots, where a single-stranded DNA forms a solid phase, are aligned,
wherein each of the spots corresponds to different combination of genetic locus and number of repeats,
wherein each of the solid phase single-stranded DNA has an STR sequence with the genetic locus and the number of repeats which are corresponding to each of the spots,
wherein the sensor is used for determining whether or not a complementary single-stranded DNA in the PCR reaction solution forms a hydrogen bond with the single-stranded DNA on each spot, and
wherein the DNA testing chip control device performs
processing of injecting the PCR reaction solution into the chamber, and
processing of determining whether or not a complementary single-stranded DNA in the PCR reaction solution forms a hydrogen bond to a single-stranded DNA on each spot.

9. (canceled)

10. The DNA testing chip according to claim 2,
wherein the single-stranded DNA on the spot comprises a primer sequence.

11. The DNA testing chip according to claim 10,
wherein the primer sequence comprises a mismatched sequence.

12. The DNA testing chip according to claim 2, further comprising
a PCR reaction tank in which a PCR reaction is performed for STR sequences of a plurality of genetic loci.

13. The DNA testing chip according to claim 3, further comprising
a PCR reaction tank in which a PCR reaction is performed for STR sequences of a plurality of genetic loci.

14. The DNA testing chip according to claim 4, further comprising
a PCR reaction tank in which a PCR reaction is performed for STR sequences of a plurality of genetic loci.

15. The DNA testing chip according to claim 10, further comprising
a PCR reaction tank in which a PCR reaction is performed for STR sequences of a plurality of genetic loci.

16. The DNA testing chip according to claim 11, further comprising
a PCR reaction tank in which a PCR reaction is performed for STR sequences of a plurality of genetic loci.

17. The DNA testing chip according to claim 12, further comprising
a DNA extraction tank in which DNA is extracted from cells of a subject.

18. The DNA testing chip according to claim 13, further comprising
a DNA extraction tank in which DNA is extracted from cells of a subject.

19. The DNA testing chip according to claim 14, further comprising
a DNA extraction tank in which DNA is extracted from cells of a subject.

20. The DNA testing chip according to claim 15, further comprising
a DNA extraction tank in which DNA is extracted from cells of a subject.

21. The DNA testing chip according to claim 16, further comprising
a DNA extraction tank in which DNA is extracted from cells of a subject.
